(19) United States
(12) Patent Application Publication
(10) Pub. No.: US 2008/0086051 A1
(43) Pub. Date: Apr. 10, 2008

(54) System, storage medium for a computer program, and method for displaying medical images

(75) Inventor: James W. Voegele, Cincinnati, OH (US)

Correspondence Address: THOMPSON HINE L.L.P.;Intellectual Property Group, P.O. BOX 8801, DAYTON, OH, 45401-8801, US

(73) Assignee: Ethicon Endo-Surgery, Inc.

(21) Appl. No.: 11/524,216

(22) Filed: Sep. 20, 2006

(51) Int. Cl. A61B 5/05

(52) U.S. Cl. 600/424

(57) ABSTRACT

A medical system includes a display monitor, a catheter insertable into a body lumen, at least one sensor attached to the catheter and adapted to provide position data, an imager adapted to provide image data of the patient, and a computer. The computer is adapted to perform steps including receiving the position data and the image data, calculating a position indication of the at-least-one sensor using at least the position data, creating an image representation of the patient using at least the image data, and displaying on the display monitor a registered overlay image using at least the image representation of the patient and the position indication of the at-least-one position sensor. A storage medium contains a program readable by a digital computer which instructs the digital computer to perform the previously described steps. A method for visualizing a position of a catheter within a patient performs the above-described steps.

### FIELD OF THE INVENTION

The present invention is related generally to medical images, and more particularly to a medical system for displaying images, to a storage medium for a computer program for displaying images, and to a method for displaying images.

### BACKGROUND OF THE INVENTION

A physician typically accesses and visualizes tissue within a patient's gastrointestinal (GI) tract with an endoscope (such as a gastroscope or a colonoscope) having a long, flexible insertion tube. For the upper GI, a physician may insert a gastroscope into the sedated patient's mouth to examine and treat tissue in the esophagus, stomach, and proximal duodenum. For the lower GI, a physician may insert a colonoscope through the sedated patient's anus to examine the rectum and colon. The images from a video camera at the distal end of the insertion tube are displayed on a monitor for use by the physician. Some endoscopes have a working channel in the insertion tube extending from a port in the handpiece to the distal portion of the insertion tube. A physician may insert medical devices into the working channel to help diagnose or treat tissue within the patient.

Imagers are known for obtaining internal images of a patient such as ultrasound images, X-ray images, computerized tomography (CT) images, positive electron emission (PET) images, magnetic resonance (MRI) images, fluoroscope images, etc. Where needed, it is known to register these images with a real world object such as by placing a marker-sensor assembly on the patient wherein the marker is visible on at least some of the images and wherein the sensor is a part of a known position sensing system such as an AC-based system available from Biosense-Webster or a DC-based system available from Ascension Technology Corporation.

Still, scientists and engineers continue to seek improved medical systems, computer programs, and methods for displaying medical images.

### SUMMARY

A first expression of an embodiment of a medical system of the invention is a medical system which includes a display monitor, a catheter, at least one sensor, an imager, and a computer. The catheter has a distal end insertable into a body lumen of a patient. The at-least-one sensor is attached to the catheter and is adapted to provide position data. The imager is adapted to provide image data of the patient. The computer is adapted to receive the position data and the image data, to calculate a position indication of the at-least-one sensor using at least the position data, to create an image representation of the patient using at least the image data, and to display on the display monitor a registered overlay image using at least the image representation of the patient and the position indication of the at-least-one position sensor.

A first expression of an embodiment of a storage medium of the invention is a storage medium which contains a program readable by a digital computer which instructs the digital computer to: receive position data from at least one sensor attached to a catheter having a distal end insertable into a body lumen of a patient; receive image data of the patient from an imager; calculate a position indication of the at-least-one sensor using at least the position data; create an image representation of the patient using at least the image data; and display on a display monitor a registered overlay image using at least the image representation of the patient and the position indication of the at-least-one position sensor.

A first expression of a method of the invention is a method for visualizing a position of a catheter within a patient and includes: receiving position data from at least one sensor attached to a catheter having a distal end insertable into a body lumen of a patient; receiving image data of the patient from an imager; calculating a position indication of the at-least-one sensor using at least the position data; creating an image representation of the patient using at least the image data; and displaying on a display monitor a registered overlay image using at least the image representation of the patient and the position indication of the at-least-one position sensor.

Several benefits and advantages are obtained from one or more expressions of the embodiment of the system, the embodiment of the storage medium, and the method of the invention. In one example, the image data includes internal image data of the patient including at least a portion of the body lumen and includes organ image data of the patient, wherein for a single sensor at the distal tip of catheter, the overlay image shows the positions of the catheter distal tip at different times along with the body lumen and internal organs allowing the physician to better articulate and guide the catheter tube in the body lumen to identify treatment sites on the other side of the body lumen. In another example, several spaced-apart sensors are attached to the catheter along the working length of the catheter allowing the entire shape of the catheter to be seen in the overlay image additionally allowing the physician to identify undesirable looping of a portion of the catheter as the portion is being pushed into the body lumen, such undesirable looping sometimes occuring during colonoscopies. In a further example, the position indication display of the at-least-one sensor shows the anatomical shape of the body lumen into which the catheter has been inserted.

### BRIEF DESCRIPTION OF THE FIGURES

FIG. 1 is a schematic view of an embodiment of a medical system of the invention, wherein a plurality of spaced-apart sensors are attached to the catheter along the working length of the catheter;

FIG. 2 is an example of a screen display of the display monitor of the medical system of FIG. 1 showing a registered overlay image including an image representation of the patient which shows a body lumen and internal body organs and including a position indication of the plurality of sensors at a particular time when the catheter has been inserted into the body lumen of the patient;

FIG. 3 is an alternate sensor-catheter embodiment wherein a single sensor is attached to the catheter proximate the distal tip of the catheter;

FIG. 4 is an example of a screen display of the display monitor of the medical system of FIG. 1 showing a registered overlay image including a torso representation of the patient and including a plurality of position indications of the single sensor of the alternate sensor-catheter embodiment of FIG. 2 calculated at different times during the insertion of the catheter into the body lumen of the patient;

FIG. 5 is a block diagram of a method of invention which, in one example, is incorporated into a program contained in a storage medium of the digital computer of the medical system of FIG. 1.

### DETAILED DESCRIPTION

Before explaining the system embodiment, the computer program steps, and the method of the present invention in detail, it should be noted that each is not limited in its application or use to the details of construction and arrangement of parts and steps illustrated in the accompanying drawings and description. The illustrative system embodiment, computer program steps, and method of the invention may be implemented or incorporated in other embodiments, computer programs, methods, variations and modifications, and may be practiced or carried out in various ways. Furthermore, unless otherwise indicated, the terms and expressions employed herein have been chosen for the purpose of describing the illustrative embodiments and method of the present invention for the convenience of the reader and are not for the purpose of limiting the invention.

It is further understood that any one or more of the following-described system embodiment, computer program steps, method, examples, etc. can be combined with any one or more of the other following-described system embodiment, computer program steps, method, examples, etc.

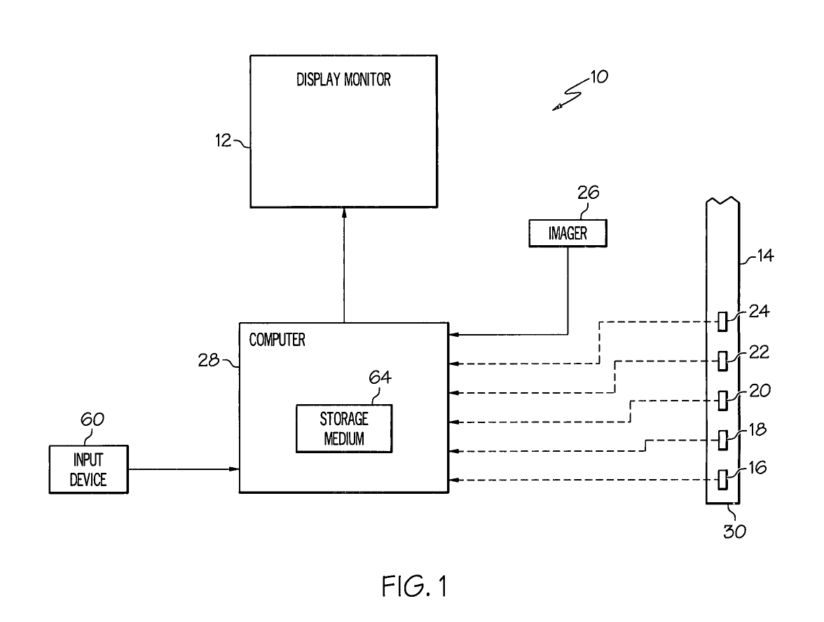

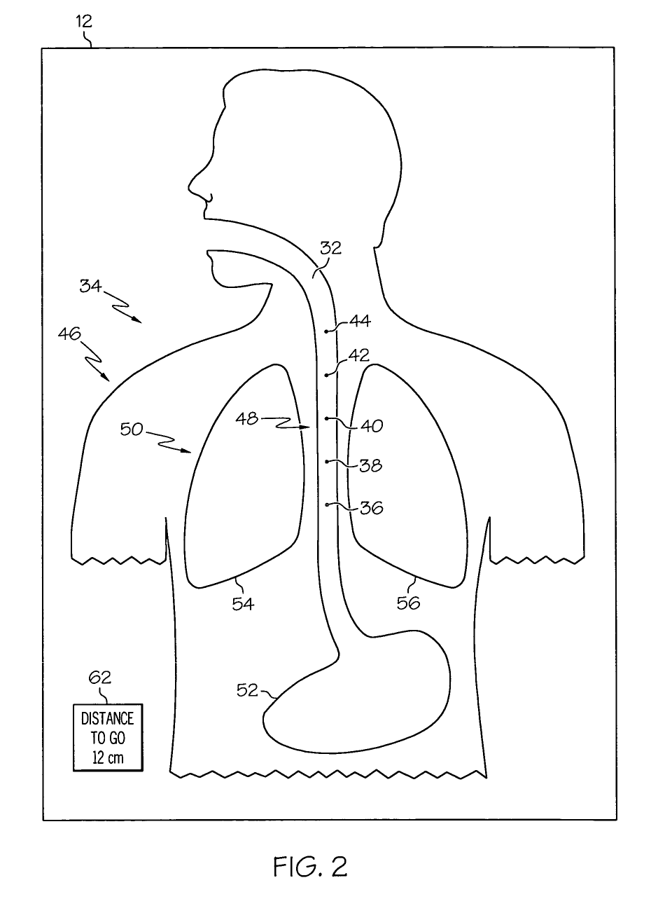

An embodiment of a medical system 10 of the invention is shown in FIGS. 1-2. A first expression of embodiment of FIGS. 1-2 is for a medical system 10 including a display monitor 12, a catheter 14, at least one sensor 16, 18, 20, 22, and 24, an imager 26, and a computer 28. The catheter 14 has a distal end 30 insertable into a body lumen 32 of a patient 34. The at-least-one sensor 16-24 is attached to the catheter 14 and is adapted to provide position data. The imager 26 is adapted to provide image data of the patient 34. The computer 28 is adapted to receive (directly or indirectly) the position data and the image data, to calculate a position indication (such as, for example, a position indicated by a small solid circle) 36, 38, 40, 42, and 44 of the at-least-one sensor 16-24 using at least the position data, to create an image representation 46 of the patient 34 using at least the image data, and to display on the display monitor 12 a registered overlay image 48 using at least the image representation 46 of the patient 34 and the position indication 36-44 of the at-least-one position sensor 16-24.

In one utilization of the first expression of the embodiment of FIGS. 1-2, the at-least-one sensor 16-24 provides the position data, the imager 26 provides the image data, and the computer 28 receives the position data and the image data, calculates the position indication 36, 38, 40, 42, and 44 of the at-least-one sensor 16-24, creates the image representation 46 of the patient 34, and displays on the display monitor 12 the registered overlay image 48. In a first variation, the time frequency, for the computer 28 to update the registered overlay image 48 displayed on the display monitor 12, is a user input to the computer. In a second variation, the time frequency is a fixed number. In a third variation, the time frequency is determined by the computer 28 based on variables such as, but not limited to, the speed of the catheter 14.

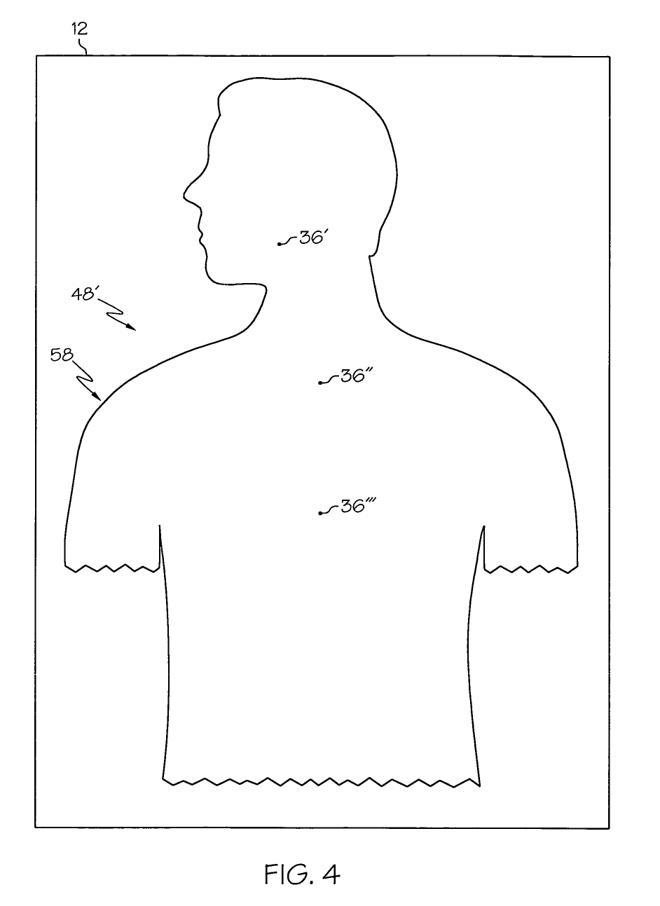

In one enablement of the first expression of the embodiment of FIGS. 1-2, the image data includes internal image data of the patient 34 describing at least a portion of the body lumen 32, and the image representation 46 is an internal image representation 50 of the patient 34 including at least the portion of the body lumen 32. In one variation, the internal image data describes a plurality of internal organs 52-56 of the patient 34 different from the body lumen 32, and the internal image representation 50 of the patient 34 includes the plurality of internal organs 52-56. In the same or a different variation, the internal image representation 50 includes the entire body lumen 32. In another enablement, as seen in the alternate embodiment of FIG. 4, the image representation 46 is a torso representation 58 of the patient 34.

In one implementation of the first expression of the embodiment of FIGS. 1-2, the catheter 14 has a working length, and the at-least-one sensor 16-24 includes a plurality of spaced apart sensors 16-24 attached to the catheter 14 along the working length of the catheter 14. In one variation, the number of sensors 16-24 is chosen allowing inter-sensor spacing to be small enough so that the position indications 36-44 displayed on the display monitor 12 look like a nearly continuous representation of the catheter 14.

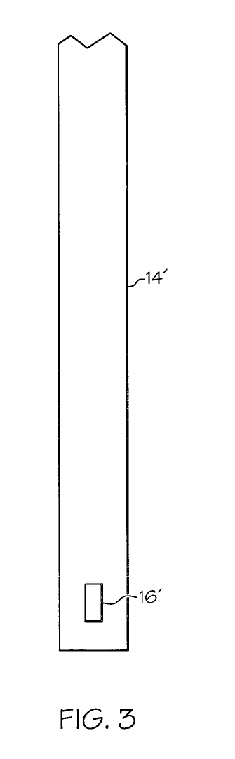

In another implementation, as shown in the alternate sensor-catheter embodiment of FIGS. 3-4, the at-least-one sensor 16′ includes exactly one sensor 16′ disposed proximate the distal end 30 of the catheter 14′. In this implementation, the computer 28 is adapted to display on the display monitor 12 a registered overlay image 48′ of the image representation 46 of the patient 34 and a plurality of position indications 36′, 36″, and 36′″ of the one sensor 16′ calculated at different times.

In an employment of the first expression of the embodiment of FIGS. 1-2, the overlay image 48 is a three-dimensional manipulative image, and the medical system 10 also includes a computer input device 60 operatively connected to the computer 28 to allow a user to manipulate the three-dimensional-manipulative image on the display monitor 12. Examples of input devices 60 include, without limitation, a keyboard and a mouse. In a different employment, as shown in the alternate embodiment of FIG. 4, the overlay image 48′ is a two-dimensional non-manipulative image.

In one extension of the first expression of the embodiment of FIGS. 1-2, the computer 28 is adapted to calculate and to display (and in one utilization calculates and displays) on the display monitor 12 at least one numerical relationship 62 derived from the position data and the image data. In one example, the at least one numerical relationship 62 includes a countdown distance remaining between the distal end 30 of the catheter 14 and a particular point along the body lumen 32, such as the end of the esophagus. In a first variation, the particular point is identified to the computer 28 by a user such as (but not limited to) a user moving a cursor over the image representation 46 of the patient 34 on the display monitor 12 and clicking on a displayed point of interest. In a second variation, the computer 28, using pattern-recognition software, identifies the particular point, such as the end of the esophagus, when (but not limited to) a user has touched “end of esophagus” from a list displayed on a touch screen portion of the display monitor 28. Other examples of numerical relationships include dimensions associated with the arcuate path of a body lumen or the catheter, catheter inserted length, and point-to-point and angular relationships of any relative features such as mouth to distal tip of catheter. Additional examples are left to the artisan.

Examples of catheters include, without limitation, cardio-vascular catheters, pulmonary catheters, and insertion tubes of endoscopes such as insertion tubes of gastroscopes and colonoscopes. Examples of body lumens of a patient include, without limitation, the upper GI (gastrointestinal) tract, the lower GI tract, and blood vessel passageways. Other examples of catheters and/or body lumens are left to the artisan.

Examples of position sensors include, without limitation, the position sensors of the AC-based position sensing system available from Biosense-Webster and the DC-based position sensing system available from Ascension Technology Corporation. It is noted in these examples, that “position” includes six degrees of freedom so that calculating position includes calculating the three-dimensional translation and the three-dimensional orientation of the position sensor with respect to a reference coordinate system. A description of the operation of such position sensors is found in US Patent Application Publication 2006/0089624.

Examples of images from imagers include, without limitation, ultrasound images, X-ray images, computerized tomography (CT) images, positive electron emission (PET) images, magnetic resonance (MRI) images, and fluoroscope images. An example of a computer program which creates a manipulative 3D display image from 2D CT-scans and MRI-scans is Mimics available from Materialise of Ann Arbor, Mich.

In one illustration of the first expression of the embodiment of FIGS. 1-2, a transmitter, not shown, of a Biosense Webster positioning sensing system is used by the computer 28 for a reference coordinate system for position data from any sensor of the Biosense Webster positioning sensing system. The sensors 16-24 of the Biosense Webster positioning sensing system which are attached to the catheter 14 provide position data of the catheter-attached sensors 16-24, and hence the catheter 14, to the computer 28. Thus, position data of the catheter 14 (i.e., the sensors 16-24 attached to the catheter) is related to the reference coordinate system. A marker-sensor assembly, not shown, is placed on the patient 34, wherein the marker portion shows up on the image data of the patient 34, is identifiable by a conventional segmentation subroutine running on the computer 28, and serves to relate the image data to the real world marker. The sensor portion of the marker-sensor assembly is another sensor of the Biosense Webster positioning sensing system and provides position data of the marker-sensor assembly to the computer. Thus, the image data is related to (the marker portion of) the marker-sensor assembly and the position of (the sensor portion of) the marker-sensor assembly is related to the reference coordinate system. This relates the image data to the reference coordinate system. As the position data of the catheter 14 (i.e., the sensors 16-24 attached to the catheter) and the image data of the patient 34 are both related to the same reference coordinate system, a subroutine can be written by those of ordinary skill in the art, without undue experimentation, which instructs the computer to create a registered overlay image of a position indication of the catheter 14 (i.e., the sensors 16-24 attached to the catheter) and an image representation of the patient 34.

A first expression of an embodiment of a storage medium 64 of the invention is a storage medium 64 containing a program readable by a digital computer 28 which instructs the digital computer 28 to perform steps a) through e). Step a) includes receiving (directly or indirectly) position data from at least one sensor 16-24 attached to a catheter 14 having a distal end 30 insertable into a body lumen 32 of a patient 34. Step b) includes receiving (directly or indirectly) image data of the patient 34 from an imager 26. Step c) includes calculating a position indication 36-44 of the at-least-one sensor 16-24 using at least the position data. Step d) includes creating an image representation 46 of the patient 34 using at least the image data. Step e) includes displaying on a display monitor 12 a registered overlay image 48 using at least the image representation 46 of the patient 34 and the position indication 36-44 of the at-least-one position sensor 16-24.

It is noted that the enablements, implementations, etc. of the previously-described first expression of the medical system 10 are equally applicable to the first expression of the embodiment of the storage medium 64. Examples of storage media include, without limitation, temporary computer memory and permanent computer memory such as RAM, hard drives, CD's, etc.

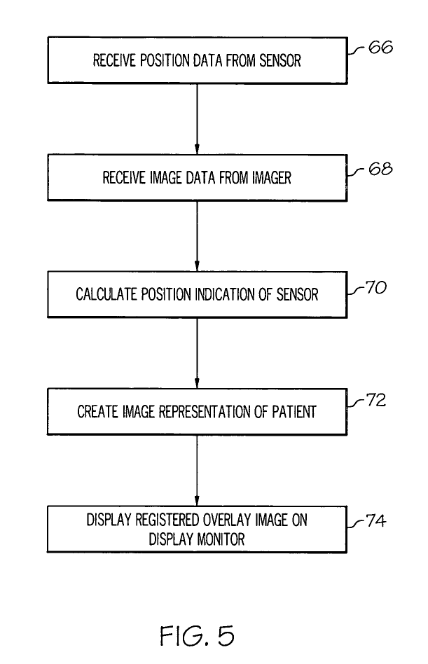

A method of the invention is for visualizing a position of a catheter 14 within a patient 34. A first expression of the method is shown in FIG. 5 and includes steps a) through e) which are identical to the previously-described steps a) through e). Step a) is labeled as “Receive Position Data From Sensor” in block 66 of FIG. 5. Step b) is labeled as “Receive Image Data From Imager” in block 68 of FIG. 5. Step c) is labeled as “Calculate Position Indication Of Sensor” in block 70 of FIG. 5. Step d) is labeled as “Create Image Representation Of Patient” in block 72 of FIG. 5. Step e) is labeled as “Display Registered Overlay Image On Display Monitor” in block 74 of FIG. 5.

It is noted that the enablements, implementations, etc. of the previously-described first expression of the medical system 10 are equally applicable to the first expression of the method.

Several benefits and advantages are obtained from one or more expressions of the embodiment of the system, the embodiment of the storage medium, and the method of the invention. In one example, the image data includes internal image data of the patient including at least a portion of the body lumen and includes organ image data of the patient, wherein for a single sensor at the distal tip of catheter, the overlay image shows the positions of the catheter distal tip at different times along with the body lumen and internal organs allowing the physician to better articulate and guide the catheter tube in the body lumen to identify treatment sites on the other side of the body lumen. In another example, several spaced-apart sensors are attached to the catheter along the working length of the catheter allowing the entire shape of the catheter to be seen in the overlay image additionally allowing the physician to identify undesirable looping of a portion of the catheter as the portion is being pushed into the body lumen, such undesirable looping sometimes occuring during colonoscopies. In a further example, the position indication display of the at-least-one sensor shows the anatomical shape of the body lumen into which the catheter has been inserted.

While the present invention has been illustrated by expressions of an embodiment, a storage medium containing a program readable by a digital computer, and a method, and enablements, implementations, etc. thereof, it is not the intention of the applicant to restrict or limit the spirit and scope of the appended claims to such detail. Numerous other variations, changes, and substitutions will occur to those skilled in the art without departing from the scope of the invention. It will be understood that the foregoing description is provided by way of example, and that other modifications may occur to those skilled in the art without departing from the scope and spirit of the appended Claims.

What is claimed is:

1. A medical system comprising:
a) a display monitor;
b) a catheter having a distal end insertable into a body lumen of a patient;
c) at least one sensor attached to the catheter and adapted to provide position data;
d) an imager adapted to provide image data of the patient; and
e) a computer adapted to receive the position data and the image data, to calculate a position indication of the at-least-one sensor using at least the position data, to create an image representation of the patient using at least the image data, and to display on the display monitor a registered overlay image using at least the image representation of the patient and the position indication of the at-least-one position sensor.

2. The medical system of claim 1, wherein the image data includes internal image data of the patient describing at least a portion of the body lumen, and wherein the image representation is an internal image representation of the patient including at least the portion of the body lumen.

3. The medical system of claim 2, wherein the internal image data describes a plurality of internal organs of the patient different from the body lumen, and wherein the internal image representation of the patient includes the plurality of internal organs.

4. The medical system of claim 3, wherein the internal image representation of the patient includes the entire body lumen.

5. The medical system of claim 1, wherein the image representation is a torso representation of the patient.

6. The medical system of claim 1, wherein the catheter has a working length, and wherein the at-least-one sensor includes a plurality of spaced apart sensors attached to the catheter along the working length of the catheter.

7. The medical system of claim 1, wherein the at-least-one sensor includes exactly one sensor disposed proximate the distal end of the catheter, and wherein the computer is adapted to display on the monitor a registered overlay image of the image representation of the patient and a plurality of position indications of the one sensor calculated at different times.

8. The medical system of claim 1, wherein the overlay image is a three-dimensional manipulative image, and also including a computer input device operatively connected to the computer to allow a user to manipulate the three-dimensional-manipulative image on the display monitor.

9. The medical system of claim 1, wherein the overlay image is a two-dimensional non-manipulative image.

10. The medical system of claim 1, wherein the computer is adapted to calculate and to display on the display monitor at least one numerical relationship derived from the position data and the image data.

11. A storage medium containing a program readable by a digital computer which instructs the digital computer to:
a) receive position data from at least one sensor attached to a catheter having a distal end insertable into a body lumen of a patient;
b) receive image data of the patient from an imager;
c) calculate a position indication of the at-least-one sensor using at least the position data;
d) create an image representation of the patient using at least the image data; and
e) display on a display monitor a registered overlay image using at least the image representation of the patient and the position indication of the at-least-one position sensor.

12. The storage medium of claim 11, wherein the image representation is a torso representation of the patient.

13. The storage medium of claim 11, wherein the image data includes internal image data of the patient describing at least a portion of the body lumen, and wherein the image representation is an internal image representation of the patient including at least the portion of the body lumen.

14. The storage medium of claim 13, wherein the internal image data describes a plurality of internal organs of the patient different from the body lumen, and wherein the internal image representation of the patient includes the plurality of internal organs.

15. The storage medium of claim 13, wherein the internal image representation of the patient includes the entire body lumen.

16. The storage medium of claim 11, wherein the at-least-one sensor includes exactly one sensor disposed proximate the distal end of the catheter, and wherein the program instructs the digital computer to display on the monitor a registered overlay image of the image representation of the patient and a plurality of position indications of the one sensor calculated at different times.

17. The medical system of claim 11, wherein the catheter has a working length, and wherein the at-least-one sensor includes a plurality of spaced apart sensors attached to the catheter along the working length of the catheter.

18. A method for visualizing a position of a catheter within a patient comprising:
a) receiving position data from at least one sensor attached to a catheter having a distal end insertable into a body lumen of a patient;
b) receiving image data of the patient from an imager;
c) calculating a position indication of the at-least-one sensor using at least the position data;
d) creating an image representation of the patient using at least the image data; and
e) displaying on a display monitor a registered overlay image using at least the image representation of the patient and the position indication of the at-least-one position sensor.

19. The method of claim 18, wherein the image representation is a torso representation.

20. The storage medium of claim 18, wherein the image data includes internal image data of the patient including at least a portion of the body lumen, and wherein the image representation is an internal image representation of the patient including at least the portion of the body lumen.
